(19) United States
(12) Patent Application Publication
(10) Pub. No.: US 2026/0027150 A1
(43) Pub. Date: Jan. 29, 2026

(54) SOLID BEVERAGE COMPOSITION AND USE METHOD THEREOF

(71) Applicant: NANJING NUTRABUILDING BIO-TECH CO., LTD., Nanjing (CN)

(72) Inventors: Long JIANG, Nanjing (CN); Xuyang SUN, Nanjing (CN); Kylin LIAO, Nanjing (CN); Shawn WELLS, Frisco, TX (US)

(21) Appl. No.: 19/343,415

(22) Filed: Sep. 29, 2025

Related U.S. Application Data
(63) Continuation of application No. PCT/CN2024/085405, filed on Apr. 2, 2024.

(30) Foreign Application Priority Data
Apr. 6, 2023 (WO) PCT/CN2023/086528

(51) Int. Cl. A61K 33/00; A23L 2/39; A23L 2/56; A23L 2/60; A23L 2/68; A61K 9/00; A61K 31/355; A61K 31/375; A61K 31/4415; A61K 31/455; A61K 31/714; A61K 33/06; A61K 33/10; A61K 33/14; A61K 33/30; A61K 47/02; A61K 47/10; A61K 47/12; A61K 47/26

(52) U.S. Cl. CPC A61K 33/00; A23L 2/39; A23L 2/56; A23L 2/60; A23L 2/68; A61K 9/0095; A61K 31/355; A61K 31/375; A61K 31/4415; A61K 31/455; A61K 31/714; A61K 33/06; A61K 33/10; A61K 33/14; A61K 33/30; A61K 47/02; A61K 47/10; A61K 47/12; A61K 47/26

(57) ABSTRACT

The invention discloses a solid beverage composition comprising by weight, 10-60% carbohydrate; 5-20% glycerol; 10-80% electrolyte; 0-8% vitamin. The osmolality of the solid beverage composition of the invention after brewing is 280-320 mosmol/kg, which can achieve ideal functions of moisture retention and loss prevention and rapid electrolyte replenishment.

### CROSS REFERENCE TO RELATED APPLICATION

This application is a continuation application of International Patent Application No. PCT/CN2024/085405, filed on Apr. 2, 2024, which claims the priority of the International Patent Application No. PCT/CN2023/086528, filed on Apr. 6, 2023, the contents of all of which are incorporated herein by reference in their entirety.

### FIELD OF THE INVENTION

The invention belongs to the technical field of food, beverage or supplement, and particularly relates to a solid beverage composition comprising by weight, 10-60% carbohydrate; 5-20% glycerol; 10-80% electrolyte; 0-8% vitamin. The osmolality of the solid beverage composition of the invention after brewing is 280-320 mosmol/kg, which can achieve ideal functions of moisture retention and loss prevention and rapid electrolyte replenishment.

### BACKGROUND OF THE INVENTION

With the continuous improvement of living standards, people consume more and more beverages. In addition, people hope that the existing drinks or beverages can achieve more comprehensive effects while supplementing body fluids and electrolytes.

Too high osmotic pressure may lead to gastric retention, nausea, vomiting and severe diarrhea in people with poor gastrointestinal function, as well as dehydration and electrolyte disorder caused by the above side effects. Too low osmotic pressure may lead to too low intestinal osmotic pressure, and intestinal water will be absorbed into intestinal wall cells, leading to edema. Therefore, while ensuring that the beverage can supplement body fluids and/or electrolytes and provide certain nutritional and health care value, it is necessary to control the beverage in the isotonic range for the beverage to be suitable for more people.

### SUMMARY OF THE INVENTION

In one aspect, the invention provides a solid beverage composition comprising by weight, 10-60% carbohydrate; 5-20% glycerol; 10-80% electrolyte; 0-8% vitamin.

In some embodiments, the carbohydrate is selected from one or more of high fructose corn syrup, sucrose, fructose, maltodextrin and glucose; the electrolyte is selected from one or more of sodium, potassium, chloride, calcium, magnesium, zinc, carbonate, phosphate, sulfate, citrate and glycinate; the vitamin is selected from one or more of vitamin A, vitamin B, vitamin C, vitamin D and vitamin E.

In some embodiments, the solid beverage composition further comprises by weight, 1-15% acidity regulator selected from one or more of citric acid, malic acid, fumaric acid, adipic acid, gluconic acid, tartaric acid, ascorbic acid, acetic acid and phosphoric acid; 0.1-4% anti-caking agent selected from one or more of tartar powder (cream of tartar), calcium silicate, silicon dioxide, microcrystalline cellulose, tricalcium phosphate and magnesium stearate; 0.1-8% flavor selected from one or more of vanilla flavor, strawberry flavor, cocoa flavor and orange flavor; 0-2% sweetener selected from one or more of Acesulfame Potassium, sucralose, mogroside, steviol glycoside, tripotassium glycyrrhizinate, sodium/calcium cyclamate, sorbitol, aspartame-acesulfame, xylitol, erythritol, maltitol and lactitol. In some embodiments, glycerol can be in the form of powder.

In some embodiments, the acidity regulator is 5-10% one or more of citric acid, malic acid, ascorbic acid, acetic acid and phosphoric acid. In some embodiments, the acidity regulator is 5-10% one or more of citric acid and malic acid.

In some embodiments, the anti-caking agent is 1-2% one or more of silicon dioxide, microcrystalline cellulose, tricalcium phosphate and magnesium stearate. In some embodiments, the anti-caking agent is one or more of silicon dioxide and microcrystalline cellulose.

In some embodiments, the solid beverage composition comprises by weight, 0.5-6% strawberry flavor, orange flavor; 0-1.5% sweetener selected from one or more of mogroside, steviol glycoside, sorbitol, xylitol, erythritol, maltitol and lactitol. In some embodiments, the flavor is strawberry flavor, orange flavor, preferably orange flavor. In some embodiments, the sweetener is mogroside, steviol glycoside, preferably steviol glycoside.

In some embodiments, the solid beverage composition comprises by weight, 15-55% carbohydrate; 8-18% glycerol; 16-62% electrolyte; 0-6% vitamin; 3-12% acidity regulator; 0.5-3% anti-caking agent. In some embodiments, the solid beverage composition comprises by weight, 20-50%, preferably 30-50% carbohydrate; 8-15%, preferably 10-15% glycerol; 18-60%, preferably 20-50.5% electrolyte; 0-5%, preferably 0-4% vitamin; 4-11%, preferably 5-10% acidity regulator; 1-3%, preferably 1-2% anti-caking agent.

In some embodiments, the carbohydrate is 30-50% one or more of glucose, sucrose and fructose. In some embodiments, the carbohydrate is glucose or sucrose, preferably glucose.

In some embodiments, the electrolyte is 20-50.5% sodium, potassium, chloride, calcium, magnesium, zinc, carbonate, citrate, glycinate or combination thereof. In some embodiments, the electrolyte is one or more of sodium chloride, sodium citrate, magnesium citrate, potassium chloride, calcium carbonate and zinc glycinate.

In some embodiments, the electrolyte is 5-10% sodium chloride, 5-10% sodium citrate, 10-20% magnesium citrate, 0-5% potassium chloride, 0-5% calcium carbonate and 0-0.5% zinc glycinate.

In some embodiments, the vitamin is 0-4% one or more of vitamin B and vitamin C. In some embodiments, the vitamin is 0-1% vitamin B3, 0-1% vitamin B6, 0-1% vitamin B12 and 0-1% vitamin C.

In some embodiments, the osmolality of the solid beverage composition after brewing is 280-320 mosmol/kg. In some embodiments, the osmolality of the solid beverage composition after brewing is 290-310 mosmol/kg.

In some embodiments, the osmolality of the solid beverage composition after brewing is 290-300 mosmol/kg.

In another aspect, the invention provides a method of using the solid beverage composition as described above, which includes administering the solid beverage composition to a subject in need thereof in an amount of 5-40 g per day. In some embodiments, the solid beverage composition is administered to a subject in need thereof in an amount of 1-50 g, 5-40 g, 8-35 g, 10-30 g, 15-20 g per day. In some embodiments, the solid beverage composition can be prepared into solid preparation or liquid preparation.

The solid beverage composition of the invention can replenish electrolytes, vitamins and the like lost due to strenuous exercise or severe diarrhea, maintain electrolyte balance, and provide high nutritional and health care values and satiety. Glycerol in the composition has strong hydration, so that it can keep human body moisture, and when compounded with electrolyte, it can simultaneously achieve ideal functions of moisture retention and loss prevention and rapid electrolyte replenishment. The composition of the invention has isotonic property and also, can greatly improve the taste and flavor.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

The inventors adjusted the composition and dosage of each component in the solid beverage composition to achieve ideal appearance, taste, balanced nutritional supplement effect and isotonic effect.

The solid beverage composition of the invention is simple and convenient to prepare, and can be prepared by uniformly mixing all the raw materials. In a preferred embodiment, the preparation method of the plant-based composition of the invention can include the following steps: firstly, uniformly mixing the raw materials with low content, and then mixing them with the raw materials with high content in equal increments. More preferably, the raw materials are mixed in a three-dimensional way for 20-30 minutes. In the preparation method of the invention, some components can be reasonably added in the above steps depending on the properties of raw materials. Considering the convenience of eating, the solid beverage composition of the invention can be packaged at 16 g per serving, and be effectively dissolved by pouring 450 mL of water during brewing. Or it can be packaged at 10 g per serving, and be effectively dissolved by pouring 280 mL of water. Or it can be packaged and blended or brewed in other suitable quantities. The osmolality after brewing is 280-320 mosmol/kg, preferably 290-300 mosmol/kg, which is in the isotonic range and is the same as the osmotic pressure of human body. It can directly supplement the loss of electrolytes in body fluids, promote the quick balance of water, salt, acid and alkali and osmotic pressure in the body, and also has strong hydration, thus maintaining human moisture, helping the human body to quickly eliminate fatigue and restore physical strength, keeping the best state, and meeting the needs of the human body. In addition, the obtained product can show balance effects of better taste, better palatability, uniform appearance, excellent stability and good solubility, and there are no problems of caking, moisture absorption and agglomeration.

The technical features, implementation methods and beneficial effects of the invention will be further described in detail with specific examples. The following examples are only part of the embodiments of the present invention, but not all of them. Unless otherwise specified, the materials and reagents described in the following examples are all common commercial products and can be purchased in the market.

### EXAMPLES

### Preparation of Solid Beverage Composition

### Example 1

The solid beverage composition of Example 1 was prepared as follows:
In step 1, vitamin C, vitamin B, sodium chloride, sodium citrate, potassium chloride, magnesium citrate and glycerol powder were sieved with a 60-mesh sieve to obtain fine powder;
In step 2, the materials sieved in step 1 were weighed, and 120 g of vitamin C, 80 g of vitamin B, 1100 g of sodium chloride, 1600 g of sodium citrate, 600 g of potassium chloride, 2100 g of magnesium citrate and 1800 g of glycerol powder were mixed;
In step 3, 6500 g of glucose was mixed with the mixed materials in step 2, and mixed with a three-dimensional mixing device for 20 min to obtain a total mixture;
In step 4, the total mixture in step 3 was internally packed with food-grade PE packaging bags at 16 g/bag;
In step 5, the inner packing bags in step 4 were externally packed with cartons.

### Example 2

The solid beverage composition of Example 2 was prepared as follows:

In step 1, vitamin C, vitamin B, vitamin E, sodium chloride, sodium citrate, potassium chloride, calcium carbonate, magnesium citrate and glycerol powder were sieved with a 60-mesh sieve to obtain fine powder;

In step 2, the materials sieved in step 1 were weighed, and 100 g of vitamin C, 50 g of vitamin B, 100 g of vitamin E, 1000 g of sodium chloride, 1200 g of sodium citrate, 500 g of potassium chloride, 300 g of calcium carbonate, 2000 g of magnesium citrate and 1800 g of glycerol powder were mixed;

In step 3, 6500 g of glucose was mixed with the mixed materials in step 2, and mixed with a three-dimensional mixing device for 20 min to obtain a total mixture;

In step 4, the total mixture in step 3 was internally packed with food-grade PE packaging bags at 16 g/bag;

In step 5, the inner packing bags in step 4 were externally packed with cartons.

### Example 3

The solid beverage composition of Example 3 was prepared as follows:
In step 1, vitamin C, vitamin E, sodium chloride, sodium citrate, magnesium citrate, zinc glycinate and glycerol powder were sieved with a 60-mesh sieve to obtain fine powder;
In step 2, the materials sieved in step 1 were weighed, and 100 g of vitamin C, 80 g of vitamin E, 800 g of sodium chloride, 1200 g of sodium citrate, 1800 g of magnesium citrate, 40 g of zinc glycinate and 1600 g of glycerol powder were mixed;
In step 3, 4500 g of fructose was mixed with the mixed materials in step 2, and mixed with a three-dimensional mixing device for 20 min to obtain a total mixture;
In step 4, the total mixture in step 3 was internally packed with food-grade PE packaging bags at 16 g/bag;
In step 5, the inner packing bags in step 4 were externally packed with cartons.

### Example 4

The solid beverage composition of Example 4 was prepared as follows:

In step 1, vitamin C, vitamin B3, vitamin B6, vitamin B12, sodium chloride, sodium citrate, potassium chloride, calcium carbonate, magnesium citrate, zinc glycinate, glycerol powder and citric acid were sieved with a 60-mesh sieve to obtain fine powder;

In step 2, the materials sieved in step 1 were weighed, and 100 g of vitamin C, 50 g of vitamin B3, 10 g of vitamin B6, 10 mg of vitamin B12, 1000 g of sodium chloride, 1500 g of sodium citrate, 500 g of potassium chloride, 300 g of calcium carbonate, 2000 g of magnesium citrate, 50 g of zinc glycinate, 2000 g of glycerol powder and 900 g of citric acid were mixed;

In step 3, 6000 g of glucose, 40 g of steviol glycoside, 200 g of silicon dioxide and 750 g of orange flavor were mixed with the mixed materials in step 2, and mixed with a three-dimensional mixing device for 20 min to obtain a total mixture;

In step 4, the total mixture in step 3 was internally packed with food-grade PE packaging bags at 16 g/bag;

In step 5, the inner packing bags in step 4 were externally packed with cartons.

### Example 5

The solid beverage composition of Example 5 was prepared as follows:

In step 1, vitamin C, vitamin B3, vitamin B6, sodium chloride, sodium citrate, potassium chloride, calcium carbonate, magnesium citrate, zinc glycinate, glycerol powder, malic acid and citric acid were sieved with a 60-mesh sieve to obtain fine powder;

In step 2, the materials sieved in step 1 were weighed, and 100 g of vitamin C, 50 g of vitamin B3, 10 g of vitamin B6, 900 g of sodium chloride, 1300 g of sodium citrate, 600 g of potassium chloride, 300 g of calcium carbonate, 1600 g of magnesium citrate, 50 g of zinc glycinate, 2000 g of glycerol powder, 100 g of malic acid and 800 g of citric acid were mixed;

In step 3, 3500 g of glucose, 1000 g of fructose, 20 g of steviol glycoside, 10 g of mogroside, 100 g of tricalcium phosphate, 100 g of magnesium stearate and 600 g of cocoa flavor were mixed with the mixed materials in step 2, and mixed with a three-dimensional mixing device for 20 min to obtain a total mixture;

In step 4, the total mixture in step 3 was internally packed with food-grade PE packaging bags at 16 g/bag;

In step 5, the inner packing bags in step 4 were externally packed with cartons.

### Example 6

The solid beverage composition of Example 6 was prepared as follows:
In step 1, vitamin C, vitamin B, sodium chloride, sodium citrate, potassium chloride, magnesium citrate, glycerol powder, malic acid and citric acid were sieved with a 60-mesh sieve to obtain fine powder;
In step 2, the materials sieved in step 1 were weighed, and 75 g of vitamin C, 80 g of vitamin B, 500 g of sodium chloride, 700 g of sodium citrate, 200 g of potassium chloride, 1000 g of magnesium citrate, 1000 g of glycerol powder, 50 g of malic acid and 450 g of citric acid were mixed;
In step 3, 3000 g of glucose, 10 g of mogroside, 100 g of tricalcium phosphate and 350 g of strawberry flavor were mixed with the mixed materials in step 2, and mixed with a biconical mixing device for 30 min to obtain a total mixture;
In step 4, the total mixture in step 3 was internally packed with food-grade PE packaging bags at 10 g/bag;
In step 5, the inner packing bags in step 4 were externally packed with cartons.

### Example 7

The solid beverage composition of Example 7 was prepared as follows:
In step 1, vitamin C, vitamin B, vitamin E, sodium chloride, sodium citrate, potassium chloride, calcium carbonate, magnesium citrate, glycerol powder and citric acid were sieved with a 60-mesh sieve to obtain fine powder;
In step 2, the materials sieved in step 1 were weighed, and 55 g of vitamin C, 90 g of vitamin B, 50 g of vitamin E, 500 g of sodium chloride, 700 g of sodium citrate, 300 g of potassium chloride, 120 g of calcium carbonate, 1000 g of magnesium citrate, 1000 g of glycerol powder and 300 g of citric acid were mixed;
In step 3, 2500 g of fructose, 100 g of microcrystalline cellulose, 350 g of orange flavor were mixed with the mixed materials in step 2, and mixed with a biconical mixing device for 30 min to obtain a total mixture;
In step 4, the total mixture in step 3 was internally packed with food-grade PE packaging bags at 10 g/bag;
In step 5, the inner packing bags in step 4 were externally packed with cartons.

### Example 8

The solid beverage composition of Example 8 was prepared as follows:
In step 1, vitamin C, vitamin E, sodium chloride, sodium citrate, magnesium citrate, zinc glycinate, glycerol powder, malic acid and citric acid were sieved with a 60-mesh sieve to obtain fine powder;
In step 2, the materials sieved in step 1 were weighed, and 60 g of vitamin C, 60 g of vitamin E, 500 g of sodium chloride, 600 g of sodium citrate, 1200 g of magnesium citrate, 20 g of zinc glycinate, 1000 g of glycerol powder, 80 g of malic acid and 420 g of citric acid were mixed;
In step 3, 2500 g of glucose, 10 g of erythritol, 100 g of tricalcium phosphate and 350 g of vanilla flavor were mixed with the mixed materials in step 2, and mixed with a biconical mixing device for 30 min to obtain a total mixture;
In step 4, the total mixture in step 3 was internally packed with food-grade PE packaging bags at 10 g/bag;
In step 5, the inner packing bags in step 4 were externally packed with cartons.

### Example 9

The solid beverage composition of Example 9 was prepared as follows:
In step 1, vitamin C, vitamin B3, vitamin B6, vitamin B12, sodium chloride, sodium citrate, potassium chloride, calcium carbonate, magnesium citrate, zinc glycinate, glycerol powder and citric acid were sieved with a 60-mesh sieve to obtain fine powder;
In step 2, the materials sieved in step 1 were weighed, and 75 g of vitamin C, 70 g of vitamin B3, 20 g of vitamin B6, 5 mg of vitamin B12, 500 g of sodium chloride, 700 g of sodium citrate, 200 g of potassium chloride, 100 g of calcium carbonate, 1000 g of magnesium citrate, 20 g of zinc glycinate, 1000 g of glycerol powder and 500 g of citric acid were mixed;
In step 3, 3000 g of glucose, 10 g of steviol glycoside, 100 g of silicon dioxide, and 350 g of strawberry flavor were mixed with the mixed materials in step 2, and mixed with a biconical mixing device for 30 min to obtain a total mixture;
In step 4, the total mixture in step 3 was internally packed with food-grade PE packaging bags at 10 g/bag;
In step 5, the inner packing bags in step 4 were externally packed with cartons.

### Example 10

The solid beverage composition of Example 10 was prepared as follows:
In step 1, vitamin C, vitamin B, vitamin E, sodium chloride, sodium citrate, potassium chloride, calcium carbonate, magnesium citrate, zinc glycinate, glycerol powder, malic acid and citric acid were sieved with a 60-mesh sieve to obtain fine powder;
In step 2, the materials sieved in step 1 were weighed, and 60 g of vitamin C, 100 g of vitamin B, 70 g of vitamin E, 400 g of sodium chloride, 600 g of sodium citrate, 200 g of potassium chloride, 100 g of calcium carbonate, 1000 g of magnesium citrate, 30 g of zinc glycinate, 1100 g of glycerol powder, 160 g of malic acid and 500 g of citric acid were mixed;
In step 3, 1600 g of fructose, 10 g of steviol glycoside, 10 g of erythritol, 90 g of silicon dioxide, 80 g of microcrystalline cellulose and 350 g of orange flavor were mixed with the mixed materials in step 2, and mixed with a biconical mixing device for 30 min to obtain a total mixture;
In step 4, the total mixture in step 3 was internally packed with food-grade PE packaging bags at 10 g/bag;
In step 5, the inner packing bags in step 4 were externally packed with cartons.

### Example 11 Property Determination

### Osmolality Test

The osmotic pressure of the solid beverage compositions of the above Examples and the competing beverages on the market was tested by the osmolality determination method of China Standard Operating Instructions for Drug Inspection (2016 edition).

Sample preparation: certain amount of samples were weighed and formulated into solutions with corresponding concentrations.

Osmotic pressure measurement: 100 μl (10%) of an above solution sample was put into a sample tube. Measuring probe of freezing point permeameter was lifted, the sample tube was put on a sample probe, with cover of the sample tube facing left. It is necessary to ensure that there is no bubble in the sample tube when adding the sample. The sample probe was pressed, and the sample name was entered. The sample was automatically determined after confirmation. Results were then printed and recorded.

The osmotic pressure data of the commercially available competing beverages (Com. Bev.) and the solid beverage compositions of the Examples of the invention are shown in the following table:

| | Recommended concentration | Osmotic pressure |
|---|---|---|
| Sample | (g/mL or g/oz) | (mosmol/kg) |
| Com. Bev. 1 | 15 g/250 mL | 196 |
| Com. Bev. 2 | 3 g/350 mL | 117 |
| Com. Bev. 3 | 16 g/16 oz (473 mL) | 246 |
| Com. Bev. 4 | 9 g/8 oz (237 mL) | 217 |
| Example 4 | 16 g/450 mL | 298 |
| Example 9 | 10 g/280 mL | 295 |

The highest osmotic pressure of the commercially available competing beverages is only 246 mosmol/kg, all of which are hypotonic. Whereas, the osmotic pressures of the solid beverage compositions of Examples 4 and 9 of the invention are 298 mosmol/kg and 295 mosmol/kg, respectively; the osmotic pressures of the solid beverage compositions of other Examples of the invention are all in the range of 280-320 mosmol/kg, even 290-300 mosmol/kg, which is isotonic, which is the same as the osmotic pressure of human body, and can directly supplement the loss of electrolytes in body fluids, and promote quick balance of water, salt, acid and alkali and osmotic pressure in the body.

### Strong Hydration Property Test

Hydration and water retention properties were tested.

The solid beverage compositions of the Examples of the invention has strong hydration and can effectively keep human body moisture.

The above are only the preferred embodiments of the present invention, and are not intended to limit the present invention. Those skilled in the art can make many changes, modifications, substitutions and variations on these embodiments without departing from the principles and purposes of the invention, and the scope of the invention is defined by the claims and their equivalents.

What is claimed is:

1. A solid beverage composition comprising by weight, 10-60% carbohydrate; 5-20% glycerol; 10-80% electrolyte; 0-8% vitamin.

2. The solid beverage composition of claim 1, wherein the carbohydrate is selected from one or more of high fructose corn syrup, sucrose, fructose, maltodextrin and glucose; the electrolyte is selected from one or more of sodium, potassium, chloride, calcium, magnesium, zinc, carbonate, phosphate, sulfate, citrate and glycinate; the vitamin is selected from one or more of vitamin A, vitamin B, vitamin C, vitamin D and vitamin E.

3. The solid beverage composition of claim 1, wherein the solid beverage composition further comprises by weight, 1-15% acidity regulator selected from one or more of citric acid, malic acid, fumaric acid, adipic acid, gluconic acid, tartaric acid, ascorbic acid, acetic acid and phosphoric acid; 0.1-4% anti-caking agent selected from one or more of tartar powder, calcium silicate, silicon dioxide, microcrystalline cellulose, tricalcium phosphate and magnesium stearate; 0.1-8% flavor selected from one or more of vanilla flavor, strawberry flavor, cocoa flavor and orange flavor; 0-2% sweetener selected from one or more of Acesulfame Potassium, sucralose, mogroside, steviol glycoside, tripotassium glycyrrhizinate, sodium/calcium cyclamate, sorbitol, aspartame-acesulfame, xylitol, erythritol, maltitol and lactitol.

4. The solid beverage composition of claim 3, wherein the acidity regulator is 5-10% one or more of citric acid, malic acid, ascorbic acid, acetic acid and phosphoric acid.

5. The solid beverage composition of claim 3, wherein the anti-caking agent is 1-2% one or more of silicon dioxide, microcrystalline cellulose, tricalcium phosphate and magnesium stearate.

6. The solid beverage composition of claim 3, wherein the solid beverage composition comprises by weight, 0.5-6% strawberry flavor, orange flavor; 0-1.5% sweetener selected from one or more of mogroside, steviol glycoside, sorbitol, xylitol, erythritol, maltitol and lactitol.

7. The solid beverage composition of claim 1, wherein the solid beverage composition comprises by weight, 15-55% carbohydrate; 8-18% glycerol; 16-62% electrolyte; 0-6% vitamin; 3-12% acidity regulator; 0.5-3% anti-caking agent.

8. The solid beverage composition of claim 1, wherein the carbohydrate is 30-50% one or more of glucose, sucrose and fructose.

9. The solid beverage composition of claim 1, wherein the electrolyte is 20-50.5% sodium, potassium, chloride, calcium, magnesium, zinc, carbonate, citrate, glycinate or combination thereof.

10. The solid beverage composition of claim 9, wherein the electrolyte is 5-10% sodium chloride, 5-10% sodium citrate, 10-20% magnesium citrate, 0-5% potassium chloride, 0-5% calcium carbonate and 0-0.5% zinc glycinate.

11. The solid beverage composition of claim 1, wherein the vitamin is 0-1% vitamin B3, 0-1% vitamin B6, 0-1% vitamin B12 and 0-1% vitamin C.

12. The solid beverage composition of claim 1, wherein the osmolality of the solid beverage composition after brewing is 280-320 mosmol/kg.

13. The solid beverage composition of claim 12, wherein the osmolality of the solid beverage composition after brewing is 290-300 mosmol/kg.
